(19) United States
(12) Patent Application Publication
(10) Pub. No.: US 2015/0182410 A1
(43) Pub. Date: Jul. 2, 2015

(54) PNEUMATIC WRAP COMPRESSION TREATMENTS FOR VENOUS CONDITIONS

(71) Applicants: Joshua Cornelius Burris, Rosenberg, TX (US); Joel Christian Burris, Rosenberg, TX (US); Jared Christopher Burris, Richmond, TX (US)

(72) Inventors: Joshua Cornelius Burris, Rosenberg, TX (US); Joel Christian Burris, Rosenberg, TX (US); Jared Christopher Burris, Richmond, TX (US)

(73) Assignee: AIJ Properties, LLC, Rosenberg, TX (US)

(21) Appl. No.: 14/145,657

(22) Filed: Dec. 31, 2013

(51) Int. Cl. A61H 9/00; A61K 31/352; A61K 31/5377; A61K 31/7028; A61K 31/4365; A61K 31/353; A61K 31/7048; A61K 31/4468; A61K 31/727; A61K 31/616

(52) U.S. Cl. CPC A61H 9/00; A61K 31/727; A61K 31/352; A61K 31/5377; A61K 31/616; A61K 31/4365; A61K 31/353; A61K 31/7048; A61K 31/4468; A61K 31/7028

(57) ABSTRACT

A method of treatment of a condition such as deep vein thrombosis, arterial insufficiency, lymphedema, pulmonary embolism, and pain includes use of a pneumatic compression wrap, pump, and medicament to provide compression on the body part of a patient and a pharmaceutical beneficial in treating the condition. Deep vein thrombosis, arterial insufficiency, lymphedema, pulmonary embolism, and pain can also be treated by an electrotherapy device utilizing a hydrogel patch containing a medicament. Apparatus and systems for performing the methods of treatment can be utilized.

### FIELD

The disclosure relates generally to a medical device and medicament. The disclosure relates specifically to a compression wrap and a medicament. The disclosure also relates to an electrotherapy device and a medicament. The disclosure also relates to a peristaltic motion device and medicament.

### BACKGROUND

Compression wraps can be used to treat conditions such as thrombosis, edema, and sprains. Compression wraps are useful in treating deep vein thrombosis. Compression increases venous flow. A compression wrap can be an elastic material such as a sleeve or a sock that provide pressure upon the body part on which it is worn. A compression wrap can be a structure that comprises at least one air cell that can be filled by an electric or manual pneumatic pump.

Electrotherapy has been utilized to treat pain. Transcutaneous Electric Nerve Stimulators (TENS) devices deliver current to nerves to control pain.

Medicaments can be used to treat conditions in various delivery forms including orally, topically, transdermally, and intervenously. Being able to provide some of the treatment with a medical device in addition to a medicament may reduce the amount of medicament needed or provide increased control of the condition.

A peristaltic pump can pump a variety of fluids by compressing a tube. Waves of contraction cause the fluid to be forced along.

It would be advantageous to have a system and method that would provide a versatile combination treatment for various conditions that would benefit from compression therapy and medicaments beneficial in treating a condition. A combination treatment of peristaltic motion and a medicament would be beneficial. It would also be advantageous to have a system and method that would provide a versatile combination treatment for various conditions that would benefit from electrotherapy and medicaments beneficial in treating a condition.

### SUMMARY

An embodiment is a method of treatment comprising providing a pneumatic compression wrap to treat a condition; wherein the pneumatic compression wrap comprises an exterior surface and an interior surface; wherein the pneumatic compression wrap is connected to a pump by one or more tubes; providing at least one transdermal patch; wherein the transdermal patch comprises a medicament to treat a condition; placing the at least one transdermal patch adjacent to an interior surface of the pneumatic compression wrap; placing the pneumatic compression wrap on a body part of a patient in need of treatment, wherein the interior surface of the pneumatic compression wrap is adjacent to the skin of the patient; and utilizing the pump and tubes to enable the pneumatic compression wrap to provide compression. In another embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In another embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In yet another embodiment, the analgesic is fentanyl, and chemical modifications thereof. In an embodiment, the transdermal patch is attached to the interior surface of the pneumatic compression wrap by an adhesive. In an embodiment, the adhesive that attaches to the interior surface of the pneumatic compression wrap is covered with an adhesive cover prior to attachment. In yet another embodiment, the transdermal patch is attached to the patient by an adhesive. In an embodiment, the adhesive that attaches to the patient is covered with an adhesive cover prior to attachment. In another embodiment, the transdermal patch is attached to the interior of the pneumatic compression wrap by a hook and loop fastener. In another embodiment, the transdermal patch is attached to the interior of the pneumatic compression wrap by a pocket that contains at least a part of the transdermal patch. In an embodiment, the body part is selected from the group consisting of calf, thigh, ankle, arch of the foot, forearm, upper arm, and chest. In an embodiment, two transdermal patches are attached to the interior surface of the pneumatic compression wrap. In an embodiment, the pump provides steady compression. In another embodiment, the pump provides compression at varying pressure levels. In yet another embodiment, the pump provides compression at time intervals. In an embodiment, the transdermal patch comprising a medicament was prepared by a compounding pharmacy. The transdermal patch and pneumatic compression wrap can be used together or separately.

An apparatus for treating a condition comprising a pneumatic compression wrap for placement on a body part of a patient; at least one transdermal patch comprising a medicament adhered to an interior surface of the pneumatic compression wrap; tubing attached to the pneumatic compression wrap; and a pump connected to the tubing. In another embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In another embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In yet another embodiment, the analgesic is fentanyl, and chemical modifications thereof. In an embodiment, the transdermal patch is attached to the interior surface of the pneumatic compression wrap by an adhesive. In an embodiment, the adhesive that attaches to the interior surface of the pneumatic compression wrap is covered with an adhesive cover prior to attachment. In yet another embodiment, the transdermal patch is attached to the patient by an adhesive. In an embodiment, the adhesive that attaches to the patient is covered with an adhesive cover prior to attachment. In another embodiment, the transdermal patch is attached to the interior of the pneumatic compression wrap by a hook and loop fastener. In another embodiment, the transdermal patch is attached to the interior of the pneumatic compression wrap by a pocket that contains at least a part of the transdermal patch. In an embodiment, the body part is selected from the group consisting of calf, thigh, ankle, arch of the foot, forearm, upper arm, and chest. In an embodiment, two transdermal patches are attached to the interior surface of the pneumatic compression wrap. In an embodiment, the transdermal patch comprising a medicament was prepared by a compounding pharmacy. The transdermal patch and pneumatic compression wrap can be used together or separately.

An embodiment is a system comprising a pneumatic compression wrap for providing compression on a body part of a patient; a transdermal patch adhered to the pneumatic compression wrap to provide a medicament to the patient to treat a condition; tubing connected to the pneumatic compression wrap; and a pump connected to the tubing to provide air pressure. In another embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In another embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In yet another embodiment, the analgesic is fentanyl, and chemical modifications thereof. In an embodiment, the transdermal patch is attached to the interior surface of the pneumatic compression wrap by an adhesive. In an embodiment, the adhesive that attaches to the interior surface of the pneumatic compression wrap is covered with an adhesive cover prior to attachment. In yet another embodiment, the transdermal patch is attached to the patient by an adhesive. In an embodiment, the adhesive that attaches to the patient is covered with an adhesive cover prior to attachment. In another embodiment, the transdermal patch is attached to the interior of the pneumatic compression wrap by a hook and loop fastener. In another embodiment, the transdermal patch is attached to the interior of the pneumatic compression wrap by a pocket that contains at least a part of the transdermal patch. In an embodiment, the body part is selected from the group consisting of calf, thigh, ankle, arch of the foot, forearm, upper arm, and chest. In an embodiment, two transdermal patches are attached to the interior surface of the pneumatic compression wrap. In an embodiment, the pump provides steady compression. In another embodiment, the pump provides compression at varying pressure levels. In yet another embodiment, the pump provides compression at time intervals. In an embodiment, the transdermal patch comprising a medicament was prepared by a compounding pharmacy. The transdermal patch and pneumatic compression wrap can be used together or separately.

In an embodiment, the pneumatic compression wrap is used with at least one transdermal patch. In an embodiment, the transdermal patch comprises a hydrogel. In an embodiment, a cream comprising a medicament is rubbed on the patient's skin prior to placing the pneumatic compression wrap on the body part of the patient. In an embodiment, the transdermal patch is used without the pneumatic compression wrap. In an embodiment, the pump is a manual pump. In an embodiment, the pump is an electric pump. In an embodiment, the pneumatic compression wrap and the medicament are treating the same condition. In an embodiment, the pneumatic compression wrap and the medicament are treating different conditions. The transdermal patch and pneumatic compression wrap can be used together or separately.

In an embodiment, a method of treatment comprising providing an electrical stimuli to treat a condition; wherein the electrical stimuli is provided by an electrotherapy device; providing at least one hydrogel patch; wherein the hydrogel patch comprises a medicament to treat the condition and an adhesive; placing the at least one hydrogel patch adjacent to an electrode of the electrotherapy device; and adhering the at least one hydrogel patch and electrode on a body part of a patient in need of treatment, wherein the hydrogel is adjacent to the skin of the patient and the electrode is adjacent to the hydrogel. In another embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In another embodiment, the anticoagulant is selected from the group consisting of heparin, enoxaparin, warfarin, rivaroxaban, acetylsalicylic acid, ticlopidine, prostaglandins, and chemical modifications thereof. In yet another embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In an embodiment, the analgesic is fentanyl, and chemical modifications thereof. In an embodiment, the hydrogel patch is attached to the interior surface of the electrotherapy device by an adhesive. In an embodiment, the hydrogel portion of hydrogel patch is covered with an adhesive cover prior to attachment. Another embodiment further comprises attaching the hydrogel patch to the patient by an adhesive. In an embodiment, the adhesive that attaches to the patient is covered with an adhesive cover prior to attachment. In another embodiment, the body part is selected from the group consisting of calf, thigh, ankle, arch of the foot, forearm, upper arm, and chest. In an embodiment, two hydrogel patches are attached to the interior surface of the electrotherapy device. In an embodiment the electrotherapy device provides the electrical stimuli at varying levels. In another embodiment, the electrotherapy device provides the electrical stimuli at time intervals. In an embodiment, the hydrogel patch comprising a medicament was prepared by a compounding pharmacy. The hydrogel patch and electrical stimuli can be used together or separately.

In an embodiment, an apparatus comprising an electrical source for providing an electrical stimuli to treat a condition; at least one hydrogel patch; wherein the hydrogel patch comprises a medicament to treat the condition, an adhesive, and an electrode, wherein the electrical source is connected to the electrode. In another embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, and analgesic. In another embodiment, the anticoagulant is selected from the group consisting of heparin, enoxaparin, warfarin, rivaroxaban, acetylsalicylic acid, ticlopidine, prostaglandins, and chemical modifications thereof. In yet another embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In an embodiment, the analgesic is fentanyl, and chemical modifications thereof. In an embodiment, the hydrogel patch is attached to the interior surface of the electrotherapy device by an adhesive. In an embodiment, the hydrogel portion of hydrogel patch is covered with an adhesive cover prior to attachment. Another embodiment further comprises attaching the hydrogel patch to the patient by an adhesive. In an embodiment, the adhesive that attaches to the patient is covered with an adhesive cover prior to attachment. In another embodiment, the body part is selected from the group consisting of calf, thigh, ankle, arch of the foot, forearm, upper arm, and chest. In an embodiment, two hydrogel patches are attached to the interior surface of the electrotherapy device. In an embodiment the electrotherapy device provides the electrical stimuli at varying levels. In another embodiment, the electrotherapy device provides the electrical stimuli at time intervals. In an embodiment, the hydrogel patch comprising a medicament was prepared by a compounding pharmacy. The hydrogel patch and electrical stimuli can be used together or separately.

In an embodiment, a system comprising an electrical source for providing an electrical stimuli to treat a condition, providing at least one hydrogel patch; wherein the hydrogel patch comprises a medicament to treat the condition and an adhesive; placing the at least one hydrogel patch adjacent to an electrode of the electrotherapy device; and wherein the at least one hydrogel patch and electrode is capable of being adhered on a body part of a patient in need of treatment with the hydrogel adjacent to the skin of the patient and the electrode is adjacent to the hydrogel. In another embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, and analgesic. In another embodiment, the anticoagulant is selected from the group consisting of heparin, enoxaparin, warfarin, rivaroxaban, acetylsalicylic acid, ticlopidine, prostaglandins, and chemical modifications thereof. In yet another embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In an embodiment, the analgesic is fentanyl, and chemical modifications thereof. In an embodiment, the hydrogel patch is attached to the interior surface of the electrotherapy device by an adhesive. In an embodiment, the hydrogel portion of hydrogel patch is covered with an adhesive cover prior to attachment. Another embodiment further comprises attaching the hydrogel patch to the patient by an adhesive. In an embodiment, the adhesive that attaches to the patient is covered with an adhesive cover prior to attachment. In another embodiment, the body part is selected from the group consisting of calf, thigh, ankle, arch of the foot, forearm, upper arm, and chest. In an embodiment, two hydrogel patches are attached to the interior surface of the electrotherapy device. In an embodiment the electrotherapy device provides the electrical stimuli at varying levels. In another embodiment, the electrotherapy device provides the electrical stimuli at time intervals. In an embodiment, the hydrogel patch comprising a medicament was prepared by a compounding pharmacy. The hydrogel patch and electrical stimuli can be used together or separately.

An embodiment of the disclosure is a method of treatment comprising providing peristaltic motion to treat a condition; wherein the peristaltic motion is provided by a peristaltic device; and placing the peristaltic motion-providing surface of the peristaltic device on a body part of a patient in need of treatment. An embodiment of the disclosure further comprises providing a transdermal patch comprising an adhesive; wherein the transdermal patch comprises a medicament to treat the condition; adhering the transdermal patch to the peristaltic motion-providing surface of the peristaltic device; and placing the peristaltic motion-providing surface of the peristaltic device and transdermal patch on a body part of a patient in need of treatment, wherein the transdermal patch is adjacent to the skin of the patient. In an embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In an embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator and chemical modifications thereof. In another embodiment, the benzopyrone is selected from the group consisting of coumarin, hydroxyethylrutin, flavonoid, and chemical modifications thereof. In an embodiment, the analgesic is fentanyl and chemical modifications thereof. In an embodiment, the transdermal patch is attached to the peristaltic motion-providing surface of the peristaltic device by an adhesive. In an embodiment, the transdermal portion of transdermal patch is covered with an adhesive cover prior to attachment. In another embodiment, the body part is selected from the group consisting of calf, thigh, ankle, arch of the foot, forearm, upper arm, and chest. In an embodiment, the peristaltic device provides the peristaltic motion at varying time intervals. In an embodiment, the peristaltic device provides the peristaltic motion at varying rates. In an embodiment, transdermal patch comprising the medicament was prepared by a compounding pharmacy. The transdermal patch and peristaltic motion can be used together or separately.

An embodiment of the disclosure is a system comprising a peristaltic device; wherein the peristaltic device provides peristaltic motion-providing surface; wherein the peristaltic motion-providing surface of the peristaltic device is placed on a body part of a patient in need of treatment of a condition. An embodiment of the disclosure further comprises a transdermal patch; wherein the transdermal patch comprises a medicament to treat the condition and an adhesive; adhering the transdermal patch to the peristaltic motion-providing surface of the peristaltic device; and placing the peristaltic motion-providing surface of the peristaltic device and transdermal patch on a body part of a patient in need of treatment of a condition, wherein the transdermal patch is adjacent to the skin of the patient. In an embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In an embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator and chemical modifications thereof. In another embodiment, the benzopyrone is selected from the group consisting of coumarin, hydroxyethylrutin, flavonoid, and chemical modifications thereof. In an embodiment, the analgesic is fentanyl and chemical modifications thereof. In an embodiment, the transdermal portion of transdermal patch is covered with an adhesive cover prior to attachment. In another embodiment, the body part is selected from the group consisting of calf, thigh, ankle, arch of the foot, forearm, upper arm, and chest. In an embodiment, the peristaltic device provides the peristaltic motion at varying time intervals. In an embodiment, the peristaltic device provides the peristaltic motion at varying rates. In an embodiment, transdermal patch comprising the medicament was prepared by a compounding pharmacy. The transdermal patch and peristaltic motion can be used together or separately.

An embodiment of the disclosure is a peristaltic device to treat a condition, wherein the peristaltic device provides peristaltic motion; wherein the peristaltic device comprises a peristaltic motion-providing surface that can be placed on a body part of a patient in need of treatment. An embodiment of the disclosure further comprises a transdermal patch comprising an adhesive; wherein the transdermal patch comprises a medicament to treat the condition and is adhered to the peristaltic motion-providing surface of the peristaltic device. In an embodiment, the condition is selected from the group consisting of deep vein thrombosis, arterial insufficiency, lymphedema, pulmonary embolism, and pain. In an embodiment, the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In an embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator and chemical modifications thereof. In another embodiment, the benzopyrone is selected from the group consisting of coumarin, hydroxyethylrutin, flavonoid, and chemical modifications thereof. In an embodiment, the analgesic is fentanyl and chemical modifications thereof. In another embodiment, the body part is selected from the group consisting of calf, thigh, ankle, arch of the foot, forearm, upper arm, and chest. In an embodiment, the peristaltic device provides the peristaltic motion at varying time intervals. In an embodiment, the peristaltic device provides the peristaltic motion at varying rates. In an embodiment, the transdermal patch comprising the medicament was prepared by a compounding pharmacy. The transdermal patch and peristaltic motion can be used together or separately.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1A is a side view of a transdermal patch.

FIG. 1B is a perspective view of a transdermal patch.

FIG. 2A is a perspective view of a pneumatic compression wrap with transdermal patches adhered thereto.

FIG. 2B is a side view of a transdermal patch adhered to a pneumatic compression wrap.

FIG. 3 is a perspective view of a pneumatic compression wrap with transdermal patches adhered to its interior surface for use on a patient's calf.

FIG. 4 is a perspective view of a pneumatic compression wrap with transdermal patches adhered to its interior surface for use on a patient's thigh and calf.

FIG. 5 is a perspective view of a pneumatic compression wrap with transdermal patches prior to the transdermal patches being placed on the interior surface of the pneumatic compression wrap for use on a patient's foot.

FIG. 6 is a perspective view of a pneumatic compression wrap with transdermal patches adhered to its interior surface for use on a patient's arm.

FIG. 7A is a perspective view of an electrotherapy device with hydrogel containing a medicament.

FIG. 7B is a perspective view of an electrotherapy device with hydrogel containing a medicament adhered to a patient's calf.

FIG. 8A is a perspective view of an electrotherapy device with a hydrogel containing a medicament.

FIG. 8B is a perspective view of an electrotherapy device with a hydrogel containing a medicament.

FIG. 8C is a perspective view of a portion of an electrotherapy device with a hydrogel containing a medicament adhered to a patient's calf.

FIG. 9A is a side view of a peristaltic device and transdermal patch on the calf of a patient.

FIG. 9B is a perspective view of the rotor of the peristaltic device.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3rd Edition.

As used herein, the term, “adhesive” means and refers to a substance used for adhering objects.

As used herein, the term, “enoxaparin” means and refers to a low molecular weight heparin.

As used herein, the term “or chemical modifications thereof” means and refers to structural changes necessary to allow the medicament to be taken up by the body.

As used herein, the term, “hook and loop fastener” means and refers to one piece has tiny hooks and the other side has loops.

As used herein, the term, “D ring and strap” means and refers to a metal or plastic ring shaped like the letter D. A fabric or plastic strap is fed between the two D rings and the strap is pulled to secure.

As used herein, the term, “pocket” means and refers to a structure for containing at least part of the patch, either at the corners of the patch or the circumference of the patch except for the top.

As used herein, the term, “transdermal” means and refers to application of a medicament through the skin.

As used herein, the term, “pneumatic” means and refers to being operated by air under pressure.

As used herein, the term, “adhesive cover” means and refers to a sheet that is able to adhere to a surface. In an embodiment, the sheet is a plastic film.

As used herein, the term, “compression wrap” means and refers to a bandage or structure that provides pressure on a body part of a patient.

As used herein, the term, “compounding pharmacy” means and refers to a pharmacy that prepares a pharmaceutical as a customized treatment for the needs of the patient.

As used herein, the term, “pressure level” means and refers to the amount of mm Hg and can include no pressure.

As used herein, the term, “adjacent” means and refers to next to or touching.

As used herein, the term, “pump” means and refers to a device for providing pressure. The pump can be electric or manual.

As used herein, the term, “added” means and refers to incorporated or placed upon.

As used herein, the term, “analgesic” means and refers to a medicament that acts to relieve pain.

As used herein, the term, “electrical stimuli” means and refers to an electrical pulse.

As used herein, the term, “hydrogel” means and refers to a gel in which the liquid portion is water.

As used herein, the term, “rotor” means and refers to a rotating part of a mechanical device.

As used herein, the term, “peristaltic device” means and refers to a positive displacement device.

In deep vein thrombosis (DVT), a blood clot forms in a vein deep in the body. If it blocks the vein, redness and swelling can occur. The blood clot can break free and travel to the lungs, causing a pulmonary embolism. There are inherited factors that place an individual at risk for DVT. These factors include lack of movement, being overweight, smoking, pregnancy, age above 60 years, and injury to a vein.

Arterial insufficiency is a lack of sufficient blood flow through the arteries. One cause of arterial insufficiency is atherosclerosis. Another cause is a blood clot.

Lymphedema is a swelling that occurs in a limb or the trunk due to a blockage in the lymphatic system. Wrapping the limb moves lymph fluid toward the trunk of the body.

A pulmonary embolism is a blockage in an artery in a lung caused by a blood clot that travels from another part of the body.

Pain can result from many conditions. In an embodiment, pain is reduced by use of a pneumatic compression wrap with analgesic patched adhered to the interior surface of the wrap.

In an embodiment, the medicament to be added to the patches is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof. In an embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In an embodiment, the analgesic is fentanyl, and chemical modifications thereof.

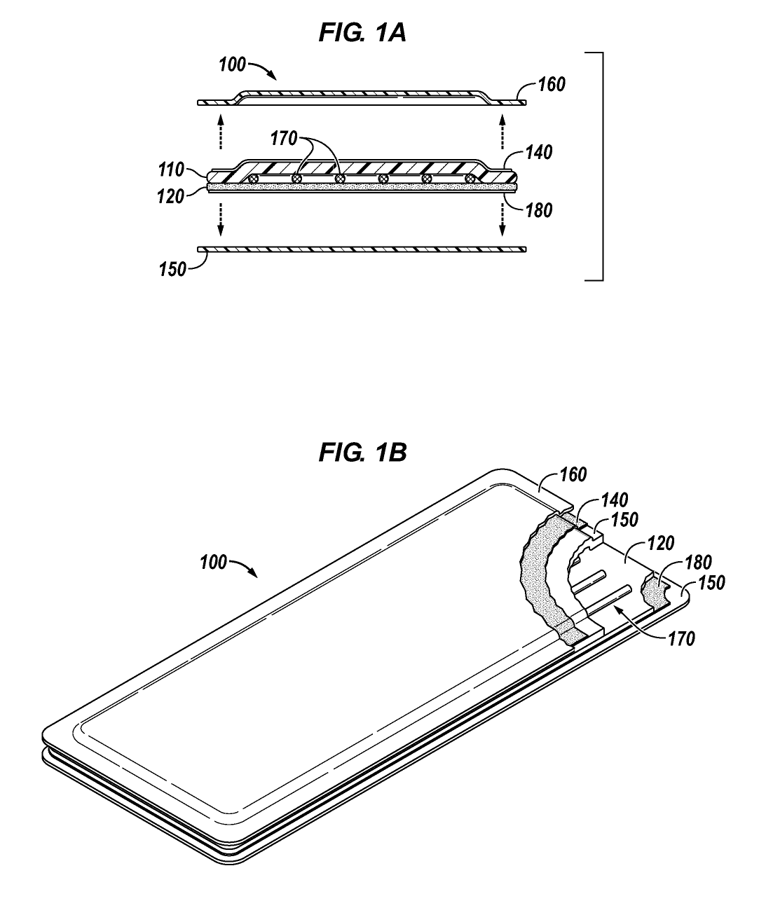

FIG. 1A depicts a side view of a transdermal patch 100. An adhesive cover 150 is adjacent to an adhesive 140 present on or near the porous membrane 120. A medicament reservoir 170 is adjacent to the porous membrane 120. A non-porous membrane 110 is present on the opposite side of the medicament reservoir 170 as the porous membrane 120. An adhesive 140 is present on the non-porous membrane 110. The adhesive 140 is adjacent to an adhesive cover 160.

FIG. 1B depicts a perspective view of a transdermal patch 100. The transdermal patch 100 is a layered structure. In an embodiment, the transdermal patch is organized in the following order from top to bottom: an adhesive cover 160, adhesive 140, non-porous membrane 110, medicament reservoir 170, porous membrane 120, adhesive 140, and adhesive cover 150.

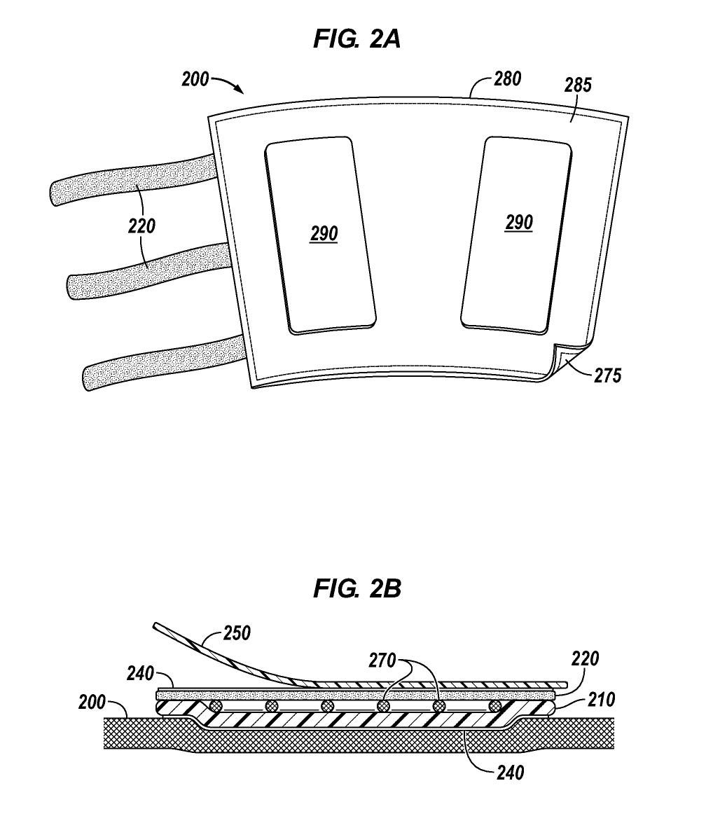

FIG. 2A depicts a perspective view of a pneumatic compression wrap 200 with transdermal patches 290 adhered thereto. The pneumatic compression wrap 200 has an exterior surface 275 and an interior surface 285. Transdermal patches 200 are adhered to the interior surface 285 of the pneumatic compression wrap 200 after removing the adhesive cover 160 from the adhesive 140 on non-porous membrane 110. The transdermal patches 200 therefore have the surface with an adhesive cover 150 adjacent to the adhesive 140 on or near the porous membrane 120 facing up.

FIG. 2B depicts a side view of a transdermal patch adhered to a pneumatic compression wrap. In an embodiment, patch and wrap are organized in the following order from top to bottom: an adhesive cover 250, adhesive 240, porous membrane 220, medicament reservoir 270, non-porous membrane 210, adhesive 240, and wrap 200.

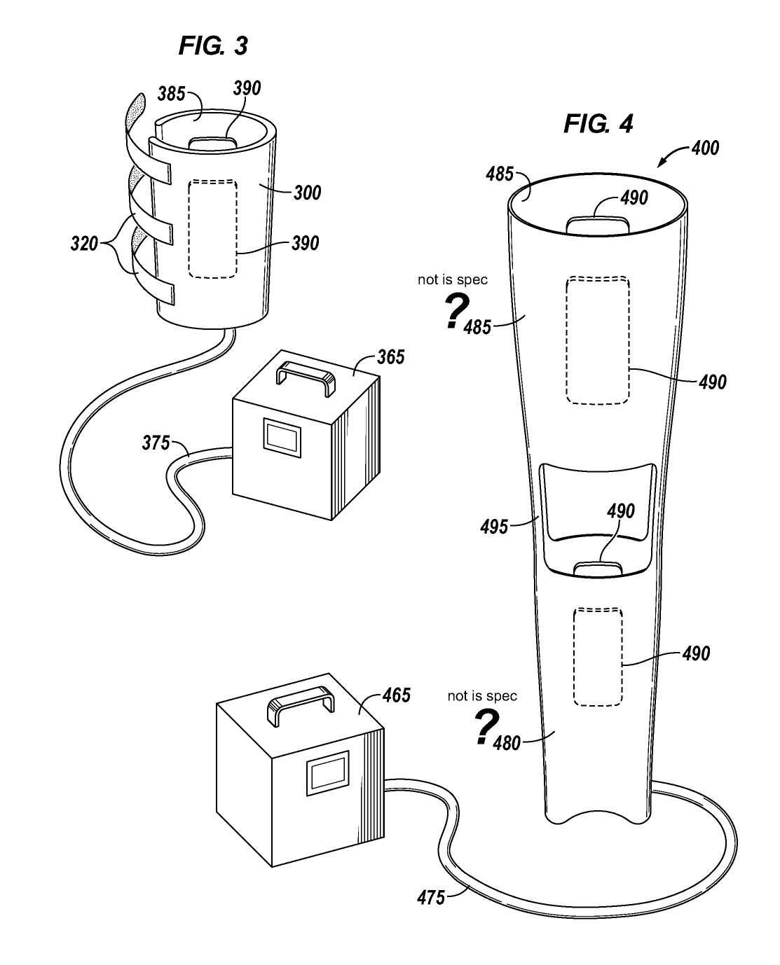

FIG. 3 depicts a perspective view of a pneumatic compression wrap 300 with transdermal patches 390 adhered to its interior surface 385 for use on a calf, with tubing 375 attached to provide air to the wrap 300. The pneumatic compression wrap 300 for a calf is a wrap 300 that has transdermal patches 390 containing a medicament reservoir 270 adhered to the interior surface 385 of the pneumatic compression wrap 300. The pneumatic compression wrap 300 comprises hook strips 320 that adhere for loop strips to fasten the pneumatic compression wrap 300 on the calf of the patient. In an embodiment, the pneumatic compression wrap 300 may cover the entire circumference of the patient's calf. In an embodiment, the pneumatic compression wrap 300 may cover a portion of the circumference of the patient's calf. In an embodiment, multiple straps may connect the sections of the pneumatic compression wrap that cover a portion of the patient's calf. The pneumatic compression wrap is connected to a pump 365 via tubing 375.

FIG. 4 depicts a perspective view of a pneumatic compression wrap 400 with transdermal patches 490 adhered to its interior surface 485 for use on a patient's thigh and calf. In an embodiment, the thigh portion of the wrap and the calf portion of the wrap are connected by a stabilizing structure 495. In an embodiment, the stabilizing structure 495 is capable of bending to allow the patient to bend the leg. In an embodiment, the pneumatic compression wrap 300 is continuous from ankle to thigh except for an opening for the patient's knee. The pneumatic compression wrap 300 is connected to a pump 465 via tubing 475.

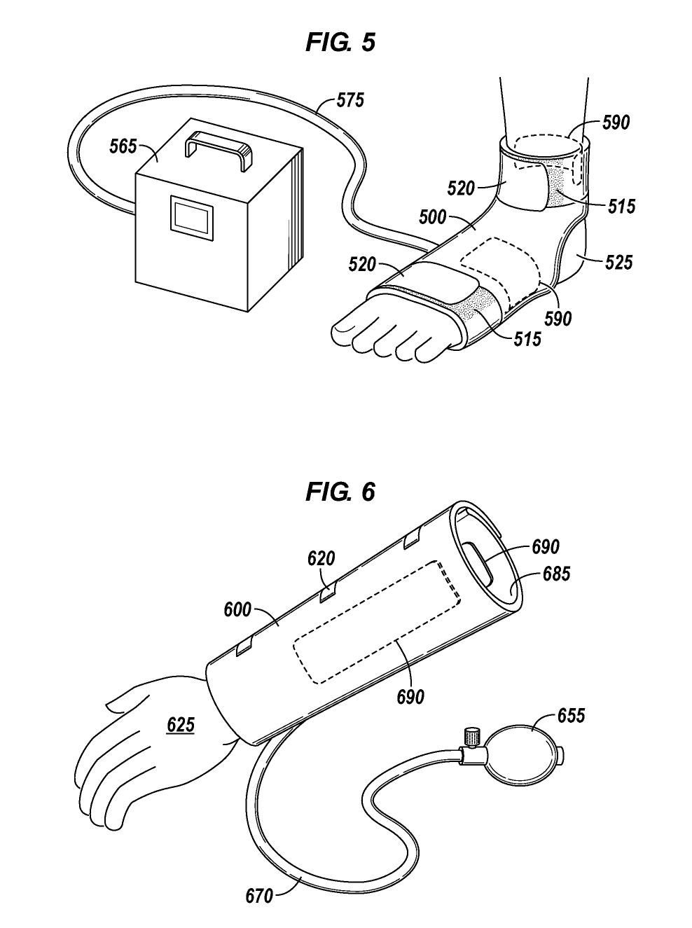

FIG. 5 depicts a perspective view of a pneumatic compression wrap 500 with transdermal patches 590 prior to the transdermal patches 590 being placed on the interior surface of the pneumatic compression wrap 500 for use on a patient's foot 525. The pneumatic compression wrap 300 is secured by at least one hook strip 520 and at least one loop strip 515. At least one transdermal patch 590 can be adhered to the interior surface of the pneumatic compression wrap 500. In an embodiment, two transdermal patches 590 are adhered to the interior surface of the pneumatic compression wrap 500. The pneumatic compression wrap 500 is connected to a pump 565 via tubing 575.

FIG. 6 depicts a perspective view of a pneumatic compression wrap 600 with transdermal patches 690 adhered to the interior surface 685 for use on a patient's arm 625. The pneumatic compression wrap 600 can be secured by at least one hook strip and at least one loop strip. In an embodiment, the pneumatic compression wrap 600 can be secured by at least one D ring and at least one strap. At least one transdermal patch 690 is adhered on the interior surface 685 of the pneumatic compression wrap 600.

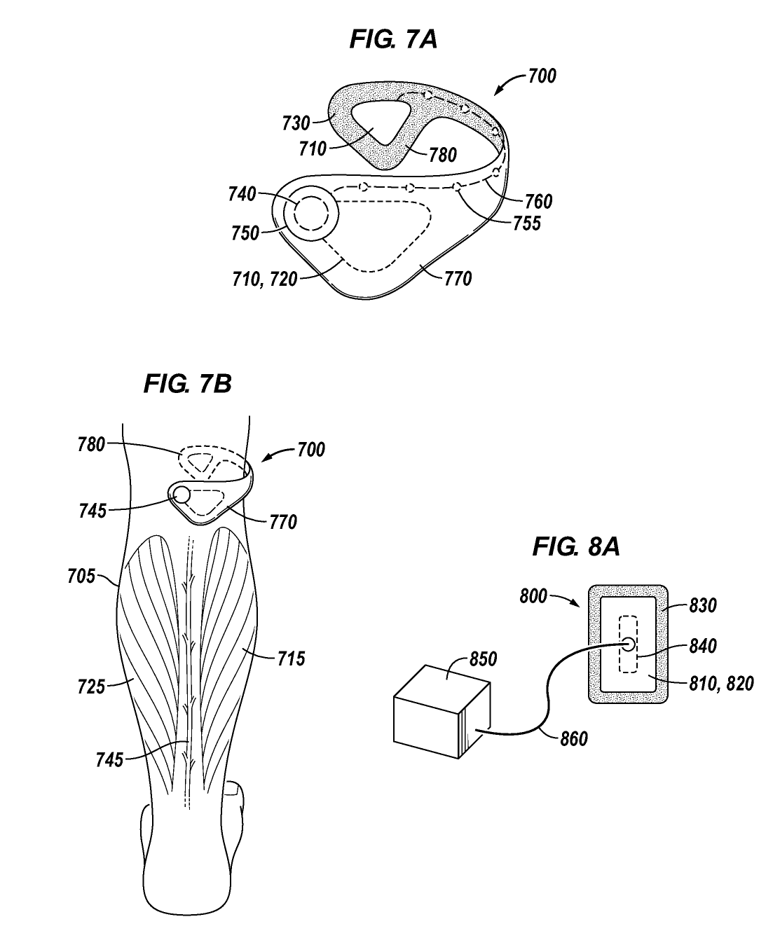

FIG. 7A is a perspective view of an electrotherapy device 700 with hydrogel 710 containing a medicament 720. The hydrogel 710 comprises a medicament 720. The hydrogel 710 is laterally surrounded by an adhesive 730. A power switch 750 is present on the electrotherapy device 700. The electrical stimulus is also applied to the muscles of the shin, including the tibialis anterior muscle. Wiring 760 connects the back extension 770 and front extension 780 to the battery 740 and electrodes 755.

FIG. 7B is a perspective view of an electrotherapy device 700 with hydrogel 710 containing a medicament 720 adhered to a patient's calf 705. The electrotherapy device 700 is adhered to the patient's calf 705. A power switch 745 is present on the back extension 770. The electrotherapy device 700 also comprises a front extension 780. The electrical stimulation of a muscle or nerve 745 caused by the electrotherapy device 700 causes the muscles of the patient, including the gastrocnemius mediale 725 and gastrocnemius laterale 715 to contract.

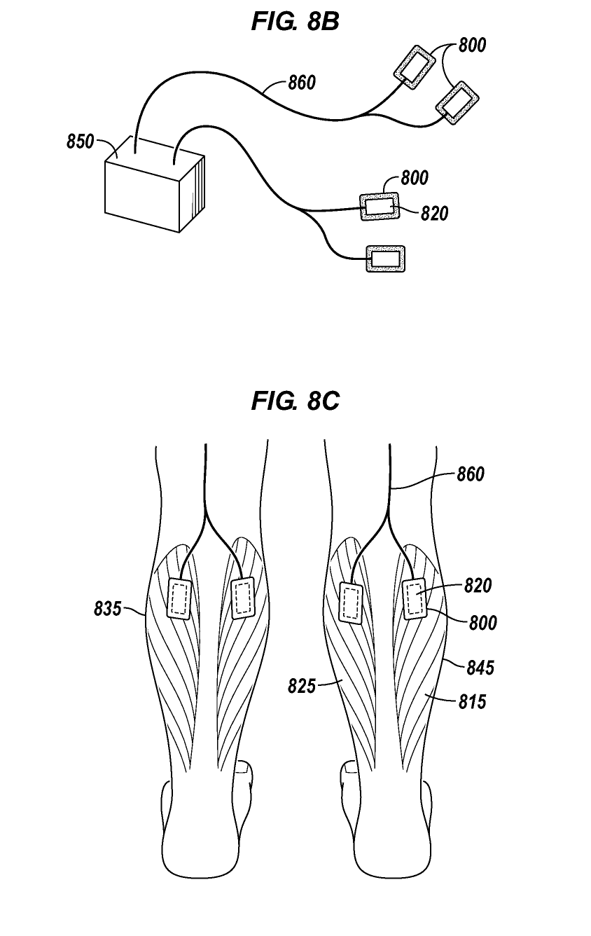

FIG. 8A is a perspective view of an electrotherapy device comprising a hydrogel patch 800, electrode 840, lead 860, and electrical source 850. The hydrogel 810 containing a medicament 820. The hydrogel patch 800 comprises an adhesive 830 around its perimeter.

FIG. 8B is a perspective view of an electrotherapy device with a hydrogel patch 800 containing a medicament 820. The hydrogel patches 800 are connected to an electrical source 850 by leads 860.

FIG. 8C is a perspective view of a portion of an electrotherapy device with a hydrogel patch 800 containing a medicament 820 adhered to a patient's left calf 835 and right calf 845. The hydrogel patches 800 are attached to an electrical source by leads 860. The hydrogel patches are adhered to the patient's left calf 835 and right calf 845 to stimulate the gastrocnemius mediale 825 and gastrocnemius laterale 815.

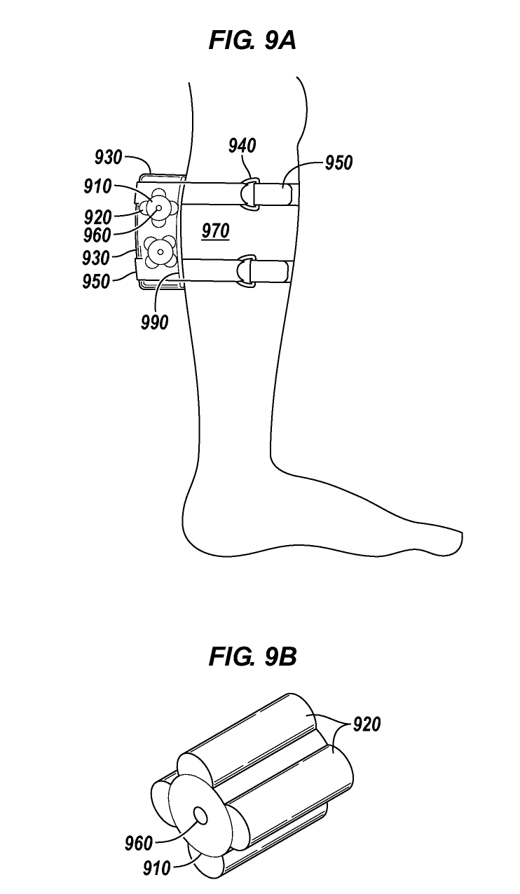

FIG. 9A is a side view of a peristaltic device 900 and transdermal patch 990 on the calf 970 of a patient. At least one lobe is present on the rotor 910. At least one rotor 910 is present in the peristaltic device 900. The at least one rotor 910 is located in a housing 930. At least one lobe 920 is present on the rotor 910. A spindle 960 can be present in the rotor 910. The peristaltic device 990 is held onto the calf 970 of the patient by D ring 940 and strap 950.

FIG. 9B is a perspective view of the rotor 910 of the peristaltic device. At least one lobe 920 is present on the rotor 910. A spindle 960 can be present in the rotor 910.

In an embodiment, a pneumatic compression wrap is made a material that is not elastic and is not air-permeable. The compression wrap can be comprised of various materials. In an embodiment, the compression wrap is comprised of urethane, cloth, and foam. In an embodiment, the interior surface of the pneumatic compression wrap is cloth. In an embodiment, the pneumatic compression wrap comprises air cells that are able to inflate and deflate with air. Pneumatic compression wraps can reduce vein volume, thus decreasing the amount of pooled blood.

Pressure is applied to the body part of the patient by air pumped into the wrap by a pump. Tubes connect the pump to the pneumatic compression wrap. In an embodiment the pump is an electric pump. In another embodiment, the pump is a hand pump.

A pneumatic compression wrap provides pressure on the leg, preventing the blood from pooling and clotting. In an embodiment, the pressure provided by the pneumatic compression wrap decreases as you approach the knee.

The pressure exerted upon the body part of the patient can be between 20 and 150 mm Hg. In an embodiment, the pressure is between 80 and 120 mm Hg. In an embodiment, the pressure is between 15 and 35 mm Hg.

In an embodiment, the pneumatic compression wrap comprises at least one air bladder. A pump provides air through a tube to expand the air bladder. The expanded air bladder provides pressure upon the body part of the patient. In an embodiment, the interior surface of the pneumatic wrap is cloth. In an embodiment, the interior surface of the pneumatic wrap is plastic. The pneumatic compression wrap can be secured on the patient by hook and loop straps that are located around the outside of the pneumatic compression wrap. In an embodiment, the pneumatic compression wrap is secured on the patient by D rings and straps. In another embodiment the pneumatic compression wrap is secured on the patient by a tight fit on the patient's body part.

In an embodiment, tubing connects the pneumatic compression wrap and a pump. The pump can be manual or electric. The tubing provides air pressure to the pneumatic compression wrap.

Transdermal gels and creams are applied directly to the skin. The stratum corneum of the skin acts as a barrier to medicament/drug molecules greater than 500 dalton. Prodrug forms of the medicament can have increased skin permeability. The medicament can be prepared in a specific manner or compounded with various substances to aid in skin penetration. In an embodiment, the medicament is in liposomes or complexed with another substance. In an embodiment, the patient's skin can be treated with modifications such as needling or microporation. In an embodiment, a gel can be used to deliver a medicament. In an embodiment a lecithin organogel can be used to deliver the medicament. A lecithin organogel can be prepared by combining lecithin, an organic solvent, and water. The medicament can penetrate the skin by the intracellular route or a transcellular route. The intracellular route is between the cells and the transcellular route is through the cells.

Transdermal patches provide a porous membrane covering a medicament reservoir. A non-porous membrane is present on top of the medicament reservoir. Once the adhesive cover present on the porous membrane is removed, the medicament can be adhered to a patient and the medicament diffuses through the porous membrane and into the skin of the patient. In an embodiment, the adhesive is present only on the periphery of the portion of the transmembrane patch that touches the patient's skin. Transdermal patches include but are not limited to reservoir, and matrix, single layer drug in an adhesive, and multi-layer drug in an adhesive. In an embodiment, the medicament in the transdermal patches can have an effect at the site of the patch or after the medicament is distributed throughout the body by the circulatory system.

In an embodiment an adhesive cover is present on both sides of transdermal patch. The adhesive cover on the surface of the transdermal patch that will be adjacent to the pneumatic compression wrap is peeled off prior to adhering the patch to the wrap. The adhesive cover of the surface of the transdermal patch that will be adjacent to the patient's skin is peeled off prior to use of the pneumatic compression wrap on the patient's body part.

The pneumatic compression wrap is interchangeable with different transdermal patches, including fresh patches of the same medicament and dose, different medicaments, or different doses. A physician can prescribe the pneumatic compression wrap and a particular dose of a medicament. In an embodiment, the transdermal patch comprising a medicament was prepared by a compounding pharmacy. In an embodiment, a compounding pharmacy thereafter compounds the desired dose of the medicament. In an embodiment, the medicament was added to the transdermal patch by a compounding pharmacy. In another embodiment, the medicament is added to the transdermal patch prior to arrival at a pharmacy. In an embodiment, the medicament can be rubbed onto the patient's skin.

In an embodiment, a method of treatment comprises providing at least one transdermal patch; wherein the transdermal patch comprises a medicament. In an embodiment, a medicament can be used to treat a patient's condition. Conditions that can be treated with the electrotherapy device and a medicament include but are not limited to deep vein thrombosis, arterial insufficiency, lymphedema, pulmonary embolism, and pain. The medicaments used to treat these conditions include but are not limited to an anticoagulant, thrombolytic, diuretic, benzopyrone, diethycarbazine, albendazole, antibiotic, antimycotic, vasodilator, rheologic agent, cannabinoid, analgesic, and chemical modifications thereof. In an embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In an embodiment, the analgesic is fentanyl, and chemical modifications thereof.

In an embodiment, the pneumatic compression wrap can be used alone to treat a condition. In another embodiment, the transdermal patch can be used alone to treat a condition.

In another embodiment, the pneumatic compression wrap and transdermal patch can be used together to treat a condition.

Stimulation of nerves and muscles causes contraction of the muscles, resulting in movement of blood toward the trunk of the body. The electrical stimuli can have varying pulse rate, pulse width, and amplitude. The frequency can range from about 1-250 hertz (Hz). In an embodiment, the frequency can range from 30 to 100 hertz. The frequency used affects which nerve groups are stimulated. The pulse width can range from about 1-300 microseconds (0). In an embodiment, the pulse width can range from 100-200 microseconds. The pulse width affects the level of muscle contraction. The amplitude can range from about 0-130 milliamps (mA). In an embodiment, the amplitude can range from 20-70 milliamps. The amplitude is how much power the device supplies and affects the strength of the tingling sensation felt by the patient.

In an embodiment, a medicament can be used in concert with the electrotherapy device to provide an additional mode of treating the patient's condition. Conditions that can be treated with the electrotherapy device and a medicament include but are not limited to deep vein thrombosis, arterial insufficiency, lymphedema, pulmonary embolism, and pain. The medicaments used to treat these conditions include but are not limited to an anticoagulant, thrombolytic, diuretic, benzopyrone, diethycarbazine, albendazole, antibiotic, antimycotic, vasodilator, rheologic agent, cannabinoid, analgesic, and chemical modifications thereof. In an embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In an embodiment, the analgesic is fentanyl, and chemical modifications thereof.

The electrotherapy device can be used on various parts of the body. In an embodiment, the body part is selected from the group consisting of calf, thigh, ankle, arch of the foot, forearm, upper arm, and chest.

The electrotherapy device is interchangeable with different hydrogel patches, including fresh patches of the same medicament and doses, different medicaments, or different doses. A physician can prescribe the electrotherapy device and a particular dose of a medicament. A compounding pharmacy thereafter compounds the desired dose of the medicament and places it in the hydrogel on the patch. In an embodiment, the hydrogel patch comprising a medicament was prepared by a compounding pharmacy. In an embodiment, a compounding pharmacy thereafter compounds the desired dose of the medicament. In an embodiment, the medicament was added to the hydrogel patch by a compounding pharmacy. In another embodiment, the medicament is added to the hydrogel patch prior to arrival at a pharmacy. In an embodiment, the medicament can be rubbed onto the patient's skin.

In an embodiment, the electrotherapy device can be used alone to treat a condition. In another embodiment, the hydrogel patch can be used alone to treat a condition. In another embodiment, the electrotherapy device and hydrogel patch can be used together to treat a condition.

In an embodiment, a medicament can be used in concert with a peristaltic device to provide an additional mode of treating the patient's condition. Conditions that can be treated with the peristaltic device and a medicament include but are not limited to deep vein thrombosis, arterial insufficiency, lymphedema, pulmonary embolism, and pain. The medicaments used to treat these conditions include but are not limited to an anticoagulant, thrombolytic, diuretic, benzopyrone, diethycarbazine, albendazole, antibiotic, antimycotic, vasodilator, rheologic agent, cannabinoid, analgesic, and chemical modifications thereof. In an embodiment, the anticoagulant is selected from the group consisting of heparin, enoxaparin, warfarin, rivaroxaban, acetylsalicylic acid, ticlopidine, prostaglandins, and chemical modifications thereof. In an embodiment, the thrombolytic is tissue plasminogen activator, and chemical modifications thereof. In an embodiment, the benzopyrone is selected from the group consisting of coumarin, hydroxyethylrutin, flavonoid, and chemical modifications thereof. In an embodiment, the analgesic is fentanyl, and chemical modifications thereof.

The peristaltic device can be used on various parts of the body. In an embodiment, the body part is selected from the group consisting of calf, thigh, ankle, arch of the foot, forearm, upper arm, and chest.

The peristaltic device is interchangeable with different transdermal patches, including fresh patches of the same medicament and doses, different medicaments, or different doses. A physician can prescribe the peristaltic device and a particular dose of a medicament. In an embodiment, the transdermal patch comprising a medicament was prepared by a compounding pharmacy. In an embodiment, a compounding pharmacy thereafter compounds the desired dose of the medicament. In an embodiment, the medicament was added to the transdermal patch by a compounding pharmacy. In another embodiment, the medicament is added to the transdermal patch prior to arrival at a pharmacy. In an embodiment, the medicament can be rubbed onto the patient's skin.

In an embodiment, the peristaltic device can be used alone to treat a condition. In another embodiment, the transdermal patch can be used alone to treat a condition. In another embodiment, the peristaltic device and transdermal patch can be used together to treat a condition.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain structures or features which are related may be substituted for the structures or features described herein while the same or similar results would be achieved. All such similar substitutes and modifications apparent to those skilled in the art are deemed to be within the spirit, scope and concept of the invention as defined by the appended claims.

1. A method of treatment comprising
providing at least one transdermal patch; wherein the transdermal patch comprises a medicament to treat a condition selected from the group consisting of deep vein thrombosis, arterial insufficiency, lymphedema, pulmonary embolism, and pain;
wherein the medicament was added to the transdermal patch by a compounding pharmacy;
providing a pneumatic compression wrap to treat a condition; wherein the pneumatic compression wrap comprises an exterior surface and an interior surface; wherein the pneumatic compression wrap is connected to a pump by one or more tubes;
wherein the pump provides compression selected from the group consisting of steady compression, compression at varying pressure levels, and compression at time intervals;
wherein the transdermal patch is attached to the interior of the pneumatic compression wrap by a pocket that contains at least a part of the transdermal patch;
wherein the transdermal patch is attached to the interior surface of the pneumatic compression wrap by one selected from the group consisting of an adhesive and at least a portion of the pneumatic compression wrap comprising a material that is non-slip; and
placing the at least one transdermal patch on a body part of a patient in need of treatment, wherein the transdermal patch is adjacent to the skin of the patient.

2. (canceled)

3. The method of claim 1 wherein the at least one transdermal patch comprises hydrogel.

4. The method of claim 1 wherein the medicament is selected from the group consisting of anticoagulant, thrombolytic, diuretic, benzopyrone, diethycarbazine, albendazole, antibiotic, antimycotic, vasodilator, rheologic agent, cannabinoid, analgesic, and chemical modifications thereof.

5. The method of claim 4 wherein the anticoagulant is selected from the group consisting of heparin, enoxaparin, warfarin, rivaroxaban, acetylsalicylic acid, ticlopidine, prostaglandins, and chemical modifications thereof.

6. The method of claim 4 wherein the thrombolytic is tissue plasminogen activator, and chemical modifications thereof.

7. The method of claim 4 wherein the benzopyrone is selected from the group consisting of coumarin, hydroxyethylrutin, flavonoid, and chemical modifications thereof.

8. The method of claim 4 wherein the analgesic is fentanyl, and chemical modifications thereof.

9. (canceled)

10. The method of claim 9 utilizing the pump and tubes to enable the pneumatic compression wrap to provide compression.

11. The method of claim 9 further comprising placing the at least one transdermal patch adjacent to an interior surface of the pneumatic compression wrap.

12. (canceled)

13. The method of claim 12 wherein the adhesive that attaches to the interior surface of the pneumatic compression wrap is covered with an adhesive cover prior to attachment.

14. The method of claim 9 further comprising attaching the at least one transdermal patch to the patient by an adhesive.

15. The method of claim 12 wherein the adhesive that attaches to the patient is covered with an adhesive cover prior to attachment.

16. The method of claim 9 wherein the transdermal patch is attached to the interior of the pneumatic compression wrap by a hook and loop fastener.

17. (canceled)

18. The method of claim 9, further comprising placing the pneumatic compression wrap on a body part of a patient in need of treatment.

19. The method of claim 18 wherein the body part is selected from the group consisting of calf, thigh, ankle, arch of the foot, forearm, upper arm, and chest.

20. (canceled)
